(19) United States
(12) Patent Application Publication
(10) Pub. No.: US 2023/0211175 A1
(43) Pub. Date: Jul. 6, 2023

(54) NANOPARTICLES, IONIZING RADIATION AND INNOVATIVE THERAPEUTIC COMBINATIONS THEREOF

(71) Applicant: NANOBIOTIX S.A., PARIS (FR)

(72) Inventors: ELSA BORGHI, PARIS (FR); LAURENT LEVY, PARIS (FR)

(21) Appl. No.: 17/927,372

(22) PCT Filed: May 25, 2021
(86) PCT No.: PCT/EP2021/063898

§ 371 Date: Nov. 23, 2022

(30) Foreign Application Priority Data
May 26, 2020 (EP) 20305548.8

(51) Int. Cl. A61N 5/10; B82Y 5/00; A61K 33/24; A61P 35/00; A61K 45/06

(52) U.S. Cl. CPC A61N 5/10; B82Y 5/00; A61K 33/24; A61P 35/00; A61K 45/06; A61N 2005/1087; A61N 2005/1098

(57) ABSTRACT

The present disclosure offers therapeutic solutions to cancer patients up to now considered as unable to undergo a standard-of-care treatment involving radiotherapy or at high risk to undergo a standard-of-care treatment involving radiotherapy. The disclosure relates to nanoparticles and/or aggregates of nanoparticles for use in the treatment of cancer in such a patient, wherein the nanoparticles and/or aggregates of nanoparticles preferably comprise more than 30% by weight of at least one chemical element having an atomic number (Z) between 20 and 83. The disclosed treatments involve a step of administering the nanoparticles and/or aggregates of nanoparticles to the patient, and a step of exposing the patient to a total dose of ionizing radiations that is equal to or less than 85% of the total dose delivered in the standard-of-care treatment. The present description also discloses new compositions comprising such nanoparticles and/or aggregates of nanoparticles as well as uses thereof.

The present invention relates to the field of human health and advantageously offers therapeutic solutions to a group of cancer patients, who, up to now, have been considered by multidisciplinary (oncology) teams (considering guidelines standards) as unable to undergo a standard-of-care treatment involving radiotherapy (RT), or, who have a high risk of intolerance to a standard-of-care treatment involving RT for their cancer. The invention, more particularly, relates to the treatment of such patients, including a step of administering nanoparticles and/or aggregates of nanoparticles preferably comprising more than 30% by weight of at least one chemical element having an atomic number (Z) between 20 and 83, and a step of exposing the patient to a total dose of ionizing radiation that is equal to or less than 85% of the total dose delivered in the standard-of-care treatment. The present description, in addition, discloses new compositions comprising such nanoparticles and/or aggregates of nanoparticles as well as uses thereof.

### BACKGROUND

In 2018, about 18.1 million people were estimated to develop cancer and there were more than 9.6 million cancer-related deaths.

Cancer may be diagnosed at various stage of the disease progression and its prognosis (i.e., the chance to recover from cancer) most often depends on the extent of disease at initial presentation. Determining the stage of cancer (“staging”) forms the basis for defining groups for inclusion in clinical trials. Most importantly, staging provides those with cancer and their physicians the critical benchmark for defining prognosis, i.e., the likelihood of overcoming the cancer, and for determining the best treatment approach for each case [see, typically, the most updated version of the American Joint Committee of Cancer (AJCC) CANCER STAGING MANUAL or the Clinical Practice Guidelines of the European Society of Medical Oncology (ESMO) for each indication].

Cancer treatment decisions are based on extensive clinical experience and may be understood as a set of treatment rules. This constitutes the “standard-of-care” therapy and is the treatment that most physicians will prescribe, that their colleagues will accept as normal, and that insurance companies usually accept for reimbursement. Standard-of-care treatment (also named “reference treatment”, “standard option”, or “treatment of choice”) is established for each indication and clinical stage.

Standard-of-care treatments are based on recommendations that typically take into account (i) the Levels of Evidence and (ii) the Grades of Recommendation and are typically presented as shown in the below Table 1.

TABLE 1
Grading of the level of clinical evidence and
strength of recommendation for clinical practice
according to the ESMO consensus guidelines
Level of evidence

| | |
|---|---|
| I | ≥1 large well-conducted randomized control trial or meta- |
| | analyses of such trials |
| II | Randomized control trials with a suspicion of bias or meta- |
| | analyses of such trials |
| III | Prospective cohort studies |
| IV | Retrospective cohort studies or case-control studies |
| V | Studies without control group, case reports, and experts' |
| | opinions |

Strength of recommendation

| | |
|---|---|
| A | Strongly recommended |
| B | Generally recommended |
| C | Optional |
| D | Generally not recommended |
| E | Never recommended |

Clinical Practice Guidelines (also considered as guidelines standards), typically edited by:

(i) the ESMO [see typically, R. Stupp et al. High-grade glioma: ESMO Clinical Practice Guidelines for diagnosis, treatment and follow-up. Annals of Oncology 25 (Supplement 3): iii93-iii101, 2014, and its updates; V Gregoire et al. Squamous cell carcinoma of the head and neck: EHNS-ESMO-ESTRO Clinical Practice Guidelines for diagnosis, treatment and follow-up. Annals of Oncology 21 (Supplement 5): v184-v186, 2010, and its updates; P. E. Postmus et al. Early and locally advanced non-small-cell lung cancer (NSCLC): ESMO Clinical Practice Guidelines for diagnosis, treatment and follow-up. Annals of Oncology 28 (Supplement 4): iv1-iv21, 2017, and its updates; F. Lordick et al. Oesophageal cancer: ESMO Clinical Practice Guidelines for diagnosis, treatment and follow-up. Annals of Oncology 27 (Supplement 5): v50-v57, 2016, and its updates; M Ducreux et al. Cancer of the pancreas: ESMO Clinical Practice Guidelines for diagnosis, treatment and follow-up. Annals of Oncology 26 (Supplement 5): v56-v68, 2015, and its updates];

(ii) the ASCO [see typically Shlomo A. Koyfman et al. Management of the Neck in Squamous Cell Carcinoma of the Oral Cavity and Oropharynx: ASCO Clinical Practice Guideline. Journal of Clinical Oncology Volume 37, Issue 20 1753, and its updates]; and

(iii) the American Society for Radiation Therapy Oncology (ASTRO) [see typically, David J. Sher et al. Radiation Therapy for Oropharyngeal Squamous Cell Carcinoma: An ASTRO Evidence Based Clinical Practice Guideline. Practical Radiation Oncology (2017), and its updates; George Rodrigues et al. Definitive and adjuvant radiotherapy in locally advanced non-small cell lung cancer: An American Society for Radiation Oncology (ASTRO) evidence-based clinical practice guideline. Practical Radiation Oncology (2015) and its updates];

give strong recommendations based on level of evidence per indication and clinical stage and are updated periodically, or when needed, to reflect the best benefit/risk ratio for the patient to be treated.

Standard-of-care treatments are also summarized in the National Comprehensive Cancer Network (NCCN) Guidelines for Patients [see, typically, NCCN Guidelines, Central Nervous System Cancers, version 1.2020; NCCN Guidelines, Esophageal and Esophagogastric Junction Cancer, Version 1.2020; NCCN Guidelines, Head and Neck Cancers, Version 1.2020; NCCN Guidelines, Non-Small Cell Lung Cancer, Version 1.2020; NCCN Guidelines, Pancreatic Adenocarcinoma, version 1.2020; NCCN Guidelines, Rectal Cancer, version 2.2020]. The NCCN (https://www.nccn.org) located at 3025 Chemical Road, Suite 100, Plymouth Meeting, PA 19462, USA, is a not-for-profit alliance of leading cancer centres devoted to patient care, research, and education. The NCCN Guidelines for Patients are drafted by an international Panel and are recognized and used worldwide by the medical profession in oncology. The NCCN guidelines are periodically updated and typically based on recommendations similar to those shown in Table 1. They are described as follows:
category 1, based upon high-level evidence with uniform NCCN consensus that the intervention is appropriate;
category 2a, based upon lower-level evidence with uniform NCCN consensus that the intervention is appropriate;
category 2b, based upon lower-level evidence with NCCN consensus that the intervention is appropriate; and
category 3, based upon any level of evidence with major NCCN disagreement that the intervention is appropriate.

The level of evidence as defined by the NCCN takes into account the quality of data, the quantity of data, and the consistency of data. The degree of consensus within the NCCN panel is based on the percentage of Panel votes.

Standard-of-care treatments for cancers include surgery, radiotherapy (RT) and/or systemic treatments. Radiotherapy (RT) is today indicated in about 50% of patients who require curative-intent treatment for solid tumors. As part of standard-of-care treatments, RT is delivered via several approaches that aim to maximize RT efficacy while minimizing damage to surrounding healthy tissue. These approaches include typically (i) biological methods, (ii) physical methods, possibly in combination with (iii) radiation modifying agents and/or (iv) immunotherapeutic agents.

The Use of Biological Methods to Optimize the Efficacy of Treatment Involving RT

Biological methods involve the delivery of fractionated radiation to leverage differences in radiation response between tumor and normal tissue, such as tumor reoxygenation, repair, redistribution of tumor cells into sensitive phases of the cell cycle, and repopulation between doses. Methods of enhancing antitumor effects may include accelerated fractionation (i.e., compared to conventional RT, generally, the same total dose is delivered but in a shortened treatment time) and hyperfractionation (i.e., compared to conventional RT, generally, a higher total dose is delivered in the same treatment time, typically twice daily), so that the killing effects on the tumor exceed those on normal tissues. Alternatively, there has been substantial interest in regimens involving a relatively large radiation dose per fraction (i.e., up to typically 20 Gy or 25 Gy) and highly conformal techniques. With these regimens, known as stereotactic body radiation therapy (SBRT) (also called stereotactic ablative radiotherapy (SABR)), ablative doses are delivered over a short period, typically, 1 to 2 weeks. The biological rationale for SBRT is complex, but presumes that the observed antitumor effect is a consequence, not only of direct tumor-cell killing, but also of indirect killing through mechanisms such as vascular collapse and immune effects.

The Use of Physical Methods to Optimize the Efficacy of Treatment Involving RT

Physical methods involve techniques to deliver a much higher dose of radiation to the tumor than to neighboring healthy tissues and/or organs at risk, for example, via a targeted image-guided treatment with intensity modulated RT (IMRT). This facilitates more precise dose escalation that is delivered to the tumor tissue only, and may preserve healthy tissues.

The Use of Radiation Modifier Agents to Optimize the Efficacy of Treatment Involving RT

An alternative method to increase the efficacy of RT is the use of radiation modifier agents that ideally selectively sensitize the tumor (vs. healthy tissues) to radiation. These agents (typically, cisplatin) ideally modify the cells by targeting cell cycle, DNA repair, or pathways known to be involved in cell survival after irradiation, so that RT acts synergistically with the radiation modifier agent(s) [cf. A. D. Colevas et al. Development of Investigational Radiation Modifiers. Journal of the National Cancer Institute, Vol. 95, No. 9, May 7, 2003].

The Use of Immunotherapeutic Agents to Optimize the Efficacy of Treatment Involving RT

More recently, the impressive successes of immunotherapy in the treatment of cancer, in particular, in the treatment of metastatic cancers, have led to tremendous excitement in the use of the combination of immunotherapy and RT.

Multimodality standard-of-care treatments involving RT are typically used for patients who have been diagnosed with locally advanced cancers.

As an example, current standard-of-care treatment options for locally advanced stage III and IV tumors in patients diagnosed with squamous cell carcinoma of the head and neck (SCCHN) are: surgery, including reconstruction, followed by postoperative RT and, for those patients found at surgery to have high-risk features, in particular, nodal extracapsular extension and/or R1 resection (i.e., corresponding to the presence of microscopic residual tumor after treatment), post-operative chemoradiotherapy (CRT) with single-agent platinum [cf. Table 1: I, A]. However, in patients with resectable tumor, when the anticipated functional outcome and/or the prognosis is so poor that potentially mutilating surgery (i.e., aggressive surgery) is not justified, combined concomitant chemoradiation is preferred. Combined concomitant chemoradiation is also the standard-of-care treatment in nonresectable patients [cf. Table 1: I, A] [cf. V. Gregoire et al. Squamous cell carcinoma of the head and neck: EHNS-ESMO-ESTRO Clinical Practice Guidelines for diagnosis, treatment and follow-up. Annals of Oncology 21 (Supplement 5): v184-v186, 2010]. Combined concomitant chemoradiation in nonresectable patients typically consists of three infusions of 100 mg/m² cisplatin given every 3 weeks, concurrently with conventionally fractionated external beam RT, typically 70 Gy, delivered with 2 Gy per fraction over 7 weeks [cf. NCCN Guidelines, Head and Neck Cancers, Version 1.2020]. However, the addition of cisplatin to RT is associated with an increase in acute adverse events, both in terms of toxicity that is related primarily to the systemic treatment (gastrointestinal, hematological, neurological, and renal side effects being observed) and in terms of toxicity owing mainly to RT itself (mucositis, dysphagia, and skin adverse events) [cf. Petr Szturz et al. Cisplatin Eligibility Issues and Alternative Regimens in Locoregionally Advanced Head and Neck Cancer: Recommendations for Clinical Practice. Frontiers in Oncology. June 2019, Volume 9, Article 464].

Radiation Enhancers

Recently, a new class of therapeutic agents—the radiation enhancer agents (also named “radioenhancer agents” or “radioenhancers”)— has emerged. Contrary to radiation modifiers, the presence of radioenhancer agents (typically, nanoparticles of high-Z elements) allows for a substantial enhancement of local radiation dose deposit at the tumor cell level (i.e., where the nanoparticles are present), when the ionizing radiation is delivered to the tumor. The goal of using such agents is to increase the energy dose deposit within the tumor mass without increasing the energy dose deposit in the surrounding healthy tissues [L. Maggiorella et al. Nanoscale radiotherapy with hafnium oxide nanoparticles. Future Oncol. (2012) 8(9), 1167-118]. Thus, radio enhancers are designed to enhance the delivery of RT precisely at the tumor site, to further optimize the benefit/risk ratio of existing treatments involving RT.

Despite the above-described efforts to optimize the benefit/risk ratio of cancer treatment and the establishment of the most appropriate treatments for patients, according to their particular cancer type and clinical stage, there remains a critical unmet need for treating patients who have been, up to now, identified as unable to undergo such standard-of-care treatments, or who are identified as being at “high risk” to undergo such standard-of-care treatments. Patients unable to undergo standard-of-care treatment are typically patients for whom the standard-of-care treatment is contraindicated, or, more broadly, who are ineligible for said treatment. Patients for whom there is a high risk of intolerance to the standard-of-care treatment thus have an unfavorable benefit/risk ratio with respect to said treatment.

For example, according to recently published clinical recommendations for systemic therapy of head and neck cancer in the elderly, “fit patients” should primarily be considered for high-dose of cisplatin with curative intent, while treatment in those who are frail rather consists of palliative measures, such as palliative irradiation and/or palliative surgical interventions (e.g., tracheostomy, gastrostomy) [cf. Petr Szturz et al. Cisplatin Eligibility Issues and Alternative Regimens in Locoregionally Advanced Head and Neck Cancer: Recommendations for Clinical Practice. Frontiers in Oncology, June 2019, Volume 9, Article 464], but these “fit patients” a priori considered for high-dose of cisplatin may be considered as a particular high-risk population in the context of the present invention as further explained herein below.

Therefore, there is a critical need to provide these patients, unable to receive a standard-of-care treatment involving RT, or at high risk to undergo a standard-of-care treatment involving RT, with an adapted treatment, typically, with a treatment having curative intent with optimum benefit/risk ratio outcome.

### SUMMARY OF THE INVENTION

Herein described are nanoparticles and/or aggregates of nanoparticles for use in the treatment of cancer in a human patient unable/ineligible to undergo a standard-of-care treatment involving RT, or in a patient at high risk of intolerance to a standard-of-care treatment involving RT, wherein the nanoparticles and/or aggregates of nanoparticles comprise more than 30% by weight of at least one chemical element having an atomic number (Z) between 20 and 83, and wherein the treatment of cancer involves a step of administering the nanoparticles and/or aggregates of nanoparticles to said patient, and a step of exposing the patient who has been administered with the nanoparticles and/or aggregates of nanoparticles to a total dose of ionizing radiations that is equal to or less than 85%, preferably, equal to or less than 80%, more preferably, equal to or less than 75%, even more preferably, equal to or less than 70%, of the total dose delivered in the standard-of-care treatment involving RT for said cancer.

In a particular aspect, the patient fulfils at least one of the following criteria:

a. is vulnerable or frail according to the comprehensive geriatric assessment (CGA),

b. has a high comorbidity index score as evaluated using the Adult Comorbidity Evaluation 27 (ACE-27) and the Charlson Comorbidity Index (CCI),

c. has grade 2 or more organ dysfunction based on the National Cancer Institute Common Toxicity Criteria (NCI CTC),

d. has poor functional status as measured as an ECOG performance status of 2 or more, or as an equivalent Karnofski performance status,

e. is exposed to more than 4 distinct prescribed drugs,

f. is elderly and evaluated as “fit”, according to part or full components evaluation of CGA, but presents cumulative exposure to risk factors for being intolerant to treatment,

g. has a locally advanced tumor observed at clinical staging,

In a particular aspect, the patient has an inoperable stage IIIA or IIIB Non-Small Cell Lung Carcinoma (NSCLC) tumor at clinical staging.

In a particular aspect, the cancer is selected from a skin cancer, a central nervous system cancer, a head and neck cancer, a lung cancer, a breast cancer, a gastrointestinal cancer, a male genitourinary cancer, a gynecologic cancer, an adrenal and/or retroperitoneal cancer, a sarcoma of bone and soft tissue, or a pediatric cancer.

In a particular aspect, the patient has a locally advanced stage III or IV squamous cell carcinoma of the head and neck (SCCHN) at clinical staging and receives a total dose of radiation of equal to or less than 59.5 Gy, preferably, equal to or less than 56 Gy, more preferably, equal to or less than 52.5 Gy, even more preferably, equal to or less than 50 Gy, delivered either as a stand-alone treatment, or in combination with chemotherapy or any other relevant systemic modality.

In a particular aspect, the patient has a stage IIIA or stage IIIB non-small cell Lung Carcinoma (NSCLC) tumor evaluated as inoperable at clinical staging, and receives a total dose of radiation equal to or less than 56.1 Gy, preferably, equal to or less than 52.8 Gy or 51 Gy, more preferably, equal to or less than 50 Gy, even more preferably, equal to or less than 49.1 Gy, 48 Gy or 42 Gy, delivered as a stand-alone treatment or in combination with chemotherapy or any other relevant systemic modality.

In a particular aspect, the patient has an operable locally advanced esophageal cancer tumor, preferably a squamous cell esophageal cancer tumor, and receives a preoperative total dose of radiation equal to or less than 35.2 Gy, preferably, equal to or less than 33.1 Gy or 31.1 Gy, more preferably, equal to or less than 30 Gy, even more preferably, equal to or less than 29 Gy, delivered as a stand-alone treatment or in combination with chemotherapy or any other relevant systemic modality.

In a particular aspect, the patient has glioblastoma and receives a total dose of radiation equal to or less than 51 Gy, preferably, equal to or less than 48 Gy, more preferably, equal to or less than 42 Gy, delivered as a stand-alone treatment or in combination with chemotherapy or any other relevant systemic modality.

Herein further described are a pharmaceutical composition comprising nanoparticles and/or aggregates of such nanoparticles and a pharmaceutically acceptable carrier or support, as well as uses thereof in a human patient unable/ineligible to undergo a standard-of-care treatment involving RT, or in human patient at high risk to intolerance to a standard-of-care treatment involving RT.

### DETAILED DESCRIPTION

The terms “cancer staging” refer to the evaluation of the extent or stage of cancer typically at the time of diagnosis. Several staging systems exist. In the “TNM” staging system, (i) the “T” refers to the size or contiguous extension of the primary tumor; (ii) the “N” refers to the absence, or presence and extent of cancer in the regional draining lymph nodes; and (iii) the “M” refers to whether the cancer has metastasized or not. The TNM system helps describe cancer in greater detail. This system classifies the extent of disease based mostly on anatomic information on the extent of the primary tumor, on the presence or absence of regional lymph nodes, and on the presence or absence of distant metastases. Still, for many cancers, the TNM combinations are grouped into five less-detailed stages, starting from stage 0 (corresponding, typically, to the presence of abnormal cells) up to stage 4 (or stage IV) (corresponding to a situation where, typically, the cancer has spread to distant parts of the body).

The terms “clinical stage” or “clinical staging” refer to the extent of disease, as defined by diagnostic study and includes any information obtained about the extent of cancer before initiation of definitive treatment. The nomenclature for clinical staging is “cT”, “cN” and “cM”, and the anatomic stage/prognostic groups based on cTNM are termed the clinical stage groups. Clinical staging incorporates information obtained from symptoms; physical examination; endoscopic examinations; imaging studies of the tumor, regional lymph nodes, and metastases; biopsies of the primary tumor; and surgical exploration without resection. When “T” is classified only clinically (“cT”), information from biopsy of single or sentinel lymph nodes may be included in clinical node staging (“cN”). On occasion, information obtained at the time of surgery may be classified as “clinical”, such as, for example, when liver metastases are identified clinically, but not biopsied during a surgical resection of an abdominal tumor.

When describing the stage of a cancer (/cancerous tumor), the words “local”, “localized”, “regional”, “locally advanced”, “distant”, “advanced” or “metastatic” may be used. The terms “local” and “localized” mean that the cancer is only in the organ where it started and has not spread to other parts of the body. “Regional” and “locally advanced” mean “close to or around the organ”. “Distant”, “advanced” and “metastatic” mean in a part of the body farther from the organ.

The term “standard-of-care” is used in the usual medical sense in the context of the invention. In the context of the invention, preferably the following combinations of Levels of Evidence and Grades of Recommendation are considered as the “standard-of-care” for a given indication by a task force composed of a panel of specialists in the field, typically a medical oncologist, a radiation oncologist, possibly along with a surgical representative specialized in an organ-specific cancer:
“I, A” and “II, A”, when referring to the ESMO Clinical Practice Guidelines (see Table 1),
“Quality of evidence: high”, “strength of recommendation: strong”, when referring to the ASTRO Clinical Practice Guidelines,
“Type: evidence based”; “Evidence quality: high, benefit outweighs harm”; “Strength of recommendation: strong”, when referring to the ASCO Clinical Practice Guidelines;
the categories “1” and “2a” when referring to the NCCN Guidelines for Patients.

The terms “treatment” or “therapy” refer to both therapeutic and prophylactic or preventive treatment or measures that can significantly slow disease progression (for example, stop tumor growth) or increase/improve Progression Free Survival (PFS) or Overall Survival (OS), or cure a cancer (i.e., turn the patient into a cancer survivor, as further defined herein below).

As described herein, such a treatment or therapy is intended for a subject in need thereof, preferably a human being, typically, a human patient identified as “unable to undergo a standard-of-care treatment involving radiotherapy” or “ineligible to undergo a standard-of-care treatment involving radiotherapy” a human patient identified as “at high risk to undergo/of intolerance to a standard-of-care treatment involving radiotherapy” (as herein explained).

In the art and in the context of the present invention, the terms “treatment having curative intent”, “curative treatment” or “curative therapy” refer to a treatment or therapy, in particular, a treatment comprising a radiotherapeutic step, offering to the subject to be treated a curative solution for treating the cancer(s) he/she is affected by, that is, for globally treating said subject [primary tumor(s) as well as corresponding metastatic lesion(s)].

As well known by the skilled person, the terms “palliative treatment” including in particular “palliative radiotherapy” are used for palliation of symptoms and are distinct from “radiotherapy”, i.e., radiotherapy delivered as curative treatment (also herein identified as “curative radiotherapy”). Indeed, palliative treatment is considered by the skilled person as an efficacious treatment for treating many symptoms induced by locally advanced or metastatic tumors, even for patients with short life expectancy.

In the context of the invention, a patient cured from its cancer is identified a “cancer survivor”. Globally, more than 33 million people are now counted as cancer survivors, and in resource-rich countries, such as the United States, extended survival means that more than 67% of patients survive more than 5 years and more than 25% of patients survive more than 15 years. Long-term cancer survivors may be considered to be ‘cured’ of their cancer [Dirk De Ruysscher et al. Radiotherapy Toxicity. Nature Reviews, 2019, 5].

In the context of the present invention, the evaluation of response criteria, including the terms “partial response” (PR), “complete response” (CR), “overall response” (OR), “best overall response” (BOR), “Stable disease” (SD) and “progressive disease” (PD), are according to the current international guidelines, for example, RECIST v1.1 guidelines as published in the European Journal of Cancer 45 (2009) (cf. pp. 228-247 “New response evaluation criteria in solid tumors: Revised RECIST guidelines (version 1.1)”).

In the context of the invention, the terms “multidisciplinary (oncology) team” (or “MDT”) refers to a cooperating group of different specialized professionals involved in cancer care with the goal of improving treatment efficiency and patient care. This team typically includes a surgeon, a medical oncologist, a radiation oncologist and/or other specialized professionals, according to patient characteristics, disease stage and indication. This team may also typically involve a pathologist, a nurse, or a hospital pharmacist. This team will typically consider guideline standards to establish the optimum treatment for the patient.

In the context of this invention, an “elderly” patient is patient aged 65 or over, classified into young-old group (from 65 to 75 years), old-old group (from 76 to 85 years), and oldest-old group (>85 years). This categorization has been adopted by the National Institute on Aging and the National Institutes of Health.

The inventors now advantageously herein describe nanoparticles and/or aggregates of nanoparticles for use in the treatment of cancer in a subject, preferably in a human patient unable to/ineligible to undergo a standard-of-care treatment involving RT or in a patient at high risk of intolerance to a standard-of-care treatment involving RT, wherein the nanoparticles and/or aggregates of nanoparticles preferably comprise more than 30% by weight of at least one chemical element having an atomic number (Z) between 20 and 83, preferably, between 40 and 83, and wherein the treatment of cancer typically involves a step of administering the nanoparticles and/or aggregates of nanoparticles to said patient, and a step of exposing the patient who has been administered with the nanoparticles and/or nanoparticles' aggregates to a total dose of ionizing radiations that is typically equal to or less than 85%, preferably, equal to or less than 80%, more preferably, equal to or less than 79%, 78%, 77%, 76% or 75%, even more preferably, equal to or less than 74%, 73%, 72%, 71% or 70%, of the total dose delivered in the standard-of-care treatment involving RT for said cancer. These nanoparticles and/or aggregates of nanoparticles significantly improve the health of the subject.

They offer, for the first time, a therapeutic solution to subjects, who, up until now, would have been considered as untreatable by multidisciplinary (oncology) teams taking into account the standard guidelines. These guidelines have been established typically by a panel of specialists in the field. Therefore, the present nanoparticles and nanoparticle aggregates offer a therapeutic solution to subjects who cannot benefit from the herein described “standard-of-care treatments”, typically, a standard-of-care treatment involving RT for said cancer.

Patient/Patients' Population Unable or at High Risk to Undergo Standard-of-Care Involving Radiotherapy (Rt)

In a first aspect of the invention, a human patient unable/ineligible to undergo a standard-of-care treatment involving RT is typically a human patient suffering of cancer who has been evaluated by a multidisciplinary (oncology) team as unable to undergo a standard-of-care treatment involving radiotherapy after determination of the benefit over risk ratio for the patient according to standard guidelines (see typically the NCCN Guidelines or the ESMO Clinical Practice Guidelines). The patient is thus considered ineligible for treatment, or unable to undergo treatment. Indeed, herein the term “unable to undergo a standard-of-care treatment involving radiotherapy” and “ineligible to undergo a standard-of-care treatment involving radiotherapy” are used interchangeably. According to certain embodiments of the invention, the standard-of-care treatment involving RT is contraindicated for said patient.

For example, surgery and/or concurrent cisplatin-RT are the current standard-of-care for most patients with a locally advanced squamous cell carcinoma (LASCC) of the head and neck. Known contraindications to cisplatin-based treatments are decreased renal function and hearing loss, for example. However, regardless of known contraindications to cisplatin-based treatment, certain patients will be considered as ineligible for (or unable to undergo) the standard-of-care treatment in the light of poor short- and/or long-term outcomes observed in clinical trials that include elderly patients and patients with poor performance status (frail patients).

According to particular aspects herein above described, this patient is, for example, an elderly patient, typically:
a “vulnerable” or “frail” patient according to the comprehensive geriatric assessment (CGA). This assessment takes into account (i) patient's inability to live independently at home or in community, as well as patient's altered physical performance, (ii) patient's inadequate social support to undergo treatment, (iii) patient's comorbidity and medication as well as patient's unfavorable nutritional status, and (iv) patient's cognition status (depression and/or anxiety diagnosis being typically considered as unfavorable). Alternatively, geriatric screening tests can be performed prior to full CGA assessment and include, among others, the G8 screening tool (typically with a cut-off value≤14 to identify vulnerable or frail cancer patient), the Flemish version of the Triage Risk Screening Tool (fTRST) (typically with a cut-off value≥1 to identify vulnerable or frail cancer patient), the Groningen Frailty Indicator (GFI) (typically with a cut-off value≥4 to identify frail cancer patient), the Vulnerable Elders Survey-13 (VES-13) (typically with a frail group (VES-13 score=3) and vulnerable group (score=1-2) identified as groups who should undergo a CGA), the mini mental state evaluation (MMSE) (typically with a cut-off value≤24 to identify impaired cognition cancer patient), the activities of daily living assessment (ADL) (typically with a cut-off value≥2-using the Katz-ADL test—to identify a cancer patient with (moderately) dependent ADL), the Instrumental Activities of Daily Living (IADL) (with typically a cut-off value≤6 to identify a cancer patient with a restriction in IADL), the Time Up and Go assessment (TUG) (with typically a cut-off value≥20 to identify a cancer patient with an impaired mobility), or an abbreviated CGA [cf. Petr Szturz et al. Treatment of elderly Patients with Squamous Cell Carcinoma of the Head and Neck. Frontiers in Oncology. August 2016, Volume 6, Article 199; Linda Bras et al. Patients with head and neck cancer: Are they frailer than patients with other solid malignancies?. Eur J Cancer Care. 2020; 29:e13170];
a patient with a high “comorbidity index score”, typically evaluated using the Adult Comorbidity Evaluation 27 (ACE-27) and the Charlson Comorbidity Index (CCI). Comorbidities are defined as additional concurrent diseases unrelated to cancer. In the context of the invention, a patient with a high comorbidity index score has typically a CCI≥2 or preferably, a CCI≥3, which defines a patient with moderate or severe comorbidities respectively [cf. Linda Bras et al. Patients with head and neck cancer: Are they frailer than patients with other solid malignancies?. Eur J Cancer Care. 2020; 29:e13170;
a patient with organ(s) dysfunction(s), typically a grade 2 or more, based on the NCI CTC (National Cancer Institute Common Toxicity Criteria—version 4.0);
a patient with a poor functional status (typically an ECOG performance status of 2 or more, or a Karnofski performance status recognized as equivalent by the skilled person, i.e., by the multidisciplinary (oncology) team [cf. Petr Szturz et al. Treatment of elderly Patients with Squamous Cell Carcinoma of the Head and Neck. Frontiers in Oncology. August 2016, Volume 6, Article 199]);
a patient with polypharmacy, typically exposed to more than four (4) distinct prescribed drugs, such polypharmacy being known to significantly increase the risk of drug-drug interactions [cf. Mostafa R. Mohamed et al. Associations of Polypharmacy and Inappropriate Medications with Adverse Outcomes in Older Adults with Cancer: A Systematic Review and Meta-Analysis. The Oncologist 2020; 25:e94-e108];
a patient with a poor nutritional status, typically evaluated through an involuntary weight loss of more than 5% typically occurring within 3 months, through a mini-nutritional assessment resulting in a score equal to or below 17, or through a body mass Index of typically less than 21 kg/m² [cf. Petr Szturz et al. Treatment of elderly Patients with Squamous Cell Carcinoma of the Head and Neck. Frontiers in Oncology. August 2016, Volume 6, Article 199; Lydia Brugel et al. Impact of comprehensive geriatric assessment on survival, function, and nutritional status in elderly patients with head and neck cancer: protocol fora multicentre randomised controlled trial (EGeSOR). BMC Cancer 2014, 14:427].

The cited guidelines are each used in their current version.

In a second aspect of the invention, a human patient at high risk to undergo a standard-of-care treatment involving radiotherapy is typically a human patient suffering from cancer who has been evaluated by a multidisciplinary (oncology) team as being at high risk to undergo a standard-of-care treatment involving RT, or in other words, as having a poor tolerance/as being at high risk of intolerance to treatment, after determination of the benefit/risk ratio for the patient according to standard guidelines (see typically the NCCN Guidelines, or the ESMO Clinical Practice Guidelines).

This patient is typically a patient evaluated as a “fit” patient, typically according to a part or full component evaluation of a CGA (i.e., a patient who is not classified as “vulnerable” or “frail”), but presenting (cumulative) exposure to risk factors. A patient presenting (cumulative) exposure to risk factors, thus considered as a patient at high risk of intolerance to treatment/as having a poor tolerance to treatment, is typically (i) a patient with physical inactivity (leading typically to overweight or obesity), smoking behavior, and/or alcohol consumption, or a patient who has been exposed to physical inactivity, smoking, and/or alcohol consumption during his lifetime (herein defined as a patient with (cumulative) exposure to an unhealthy lifestyle), and/or (ii) a patient with (cumulative) exposure to etiological agents (such as, for example, any microorganisms that can cause infection). This patient is also typically evaluated as a fit patient but with familial or hereditary risk factors.

For instance, this patient may be a fit elderly patient. As a typical example, in a combined analysis of two phase III trials conducted by ECOG (1393 and 1395), comparison of toxicity, response rates, and survival has been performed between elderly recurrent or metastatic SCCHN patients (70 years or older), and their younger counterparts. The ECOG 1393 trial randomized patients to receive a cisplatin/paclitaxel doublet at two dose levels, while treatment arms in the ECOG 1395 trial comprised cisplatin plus either 5-fluorouracil or paclitaxel. Altogether, 53 older patients were compared to 346 younger ones. No statistically significant differences were observed in terms of objective response rate (28% versus 33%), median time to progression (5.25 months versus 4.8 months), median overall survival (5.3 months versus 8 months), or 1-year survival (26% versus 33%) between these two subgroups, respectively. However, a significantly higher incidence of severe nephrotoxicity, diarrhea, and thrombocytopenia was noted in the elderly population, and was accompanied by a trend toward a higher toxic death rate (13% versus 8%). In conclusion, cisplatin-based doublets yielded comparable survival outcomes among fit elderly and younger patients, yet at the cost of increased side effects in the former group. Therefore, despite of being evaluated as fit elderly patients, a poor tolerance to treatment was noted in this patient population.

In a particular aspect, the nanoparticles and/or aggregates of nanoparticles are for use in a human patient having a locally advanced tumor, as herein above defined, observed at clinical staging.

In another particular aspect, the nanoparticles and/or aggregates of nanoparticles are for use in a human patient having a tumor evaluated as inoperable (i.e., as nonresectable) at clinical staging (because of an unfavorable benefit/risk ratio).

Total Delivered Radiation Dose

In the context of the present invention, the treatment of cancer involves a step of administering the nanoparticles and/or aggregates of nanoparticles herein described by inventors to a patient unable to/ineligible to undergo, or at high risk of intolerance to a standard-of-care treatment involving radiotherapy (RT), as evaluated typically by a multidisciplinary (oncology) team, and a step of exposing the patient who has been administered with the nanoparticles and/or nanoparticles' aggregates to a total dose of ionizing radiation that is equal to or less than 85%, preferably, equal to or less than 80%, more preferably, equal to or less than 79%, 78%, 77%, 76% or 75%, even more preferably, equal to or less than 74%, 73%, 72%, 71% or 70%, of the total dose delivered in the standard-of-care treatment involving RT for said cancer.

For example, when considering patients (or patient populations) diagnosed with locally advanced stage III or IV squamous cell carcinoma of the head and neck (SCCHN) at clinical stage, and who are ineligible for, or at high risk of intolerance, to the combined concomitant chemoradiation standard-of-care treatment option (i.e., typically, 100 mg/m² cisplatin given every 3 weeks, concurrently with conventionally fractionated external beam radiotherapy, typically 70 Gy, delivered with 2 Gy per fraction over 7 weeks), the present invention now offers an advantageous treatment solution. The RT may be given via an external beam at a total dose of ionizing radiation that is equal to or less than 85%, preferably equal to or less than 80%, more preferably, equal to or less than 75%, even more preferably, equal to or less than 74%, 73%, 72%, 71% or 70% of the total dose delivered in the standard-of-care treatment involving radiotherapy.

In a particular embodiment, the external beam radiotherapy may be given as 2 Gy per fraction (five days per week) and at a total dose of ionizing radiation that is equal to or less than 59.5 Gy, preferably, equal to or less than 56 Gy, more preferably, equal to or less than 52.5 Gy, even more preferably, equal to or less than 50 Gy, as compared to the total dose delivered in the standard-of-care treatment involving RT (given at a total dose of 70 Gy, 2 Gy per fraction over 7 weeks). This reduced ionizing radiation dose can typically be delivered as a stand-alone treatment, or in combination with chemotherapy or any other relevant systemic modality (in other words, in combination with a chemotherapeutic agent or any other relevant systemic agent) as appreciated by the skilled person.

Moreover, in head and neck cancers, the relationship between the RT dose received by the pharyngeal constrictors (the base of tongue and supraglottic larynx) and long-term swallowing dysfunction is well documented. Dysphagia increases with every 10 Gy above 55 Gy given to the superior and middle pharyngeal constrictors. Stricture and feeding tube dependence increase when the volume of pharyngeal constrictors receiving 70 Gy exceeds 50% and 30%, respectively, and aspiration increases when more than 50% of pharyngeal constrictors receive 65 Gy [H. Mirghani et al. Treatment de-escalation for HPV-driven oropharyngeal cancer: Where do we stand? Clinical and Translational Radiation Oncology 8 (2018) 4-11]. Therefore, the present invention now proposes a therapeutic solution without equivalent to these patients identified as unable, or at high risk of intolerance to the standard-of-care involving RT. This is due to the possibility to reduce the total dose of RT so that it is compatible with the treatment of these patients by reducing treatment toxicity, while maintaining therapeutic efficacy.

According to an embodiment of the invention, the patient has stage III or IV SCCHN and is ineligible for the standard-of-care treatment involving administration of a cytotoxic drug (for example cis-platin) combined with RT (usually at a total dose of 70 Gy).

According to an embodiment of the invention, the patient has stage III or IV SCCHN and is ineligible for the standard-of-care treatment involving administration of an immunotherapeutic agent (for example cetuximab) combined with RT (usually at a total dose of 70 Gy).

For patients diagnosed with/suffering from locally advanced Non-Small Cell Lung Carcinoma (NSCLC), concurrent CRT (ChemoRadioTherapy) is the treatment of choice in patients evaluated as unresectable in stage IIIA or IIIB [cf. Table 1: I, A] at clinical stage. If concurrent CRT is not possible, for any reason, sequential Chemotherapy followed by definitive RT represents a valid and effective alternative [cf. Table 1: I, A]. In the absence of contraindications, the optimal chemotherapeutic agent to be combined with exposition to radiation in stage III NSCLC is generally cisplatin with 60 Gy or 66 Gy in 30 or 33 daily fractions of ionizing radiations being recommended for concurrent CRT [cf. Table 1: I, A] [cf. P. E. Postmus et al. Early and locally advanced non-small-cell lung cancer (NSCLC): ESMO Clinical Practice Guidelines for diagnosis, treatment and follow-up. Annals of Oncology 28 (Supplement 4): iv1iv21, 2017; NCCN Guidelines, Non-Small Cell Lung Cancer, Version 1.2020].

Typically, when considering these NSCLC patients (or patient population) diagnosed in stage IIIA and IIIB with unresectable tumor (i.e., evaluated as inoperable at clinical staging) and unable/ineligible to receive, or at high risk of intolerance to the chemoradiation given as the standard-of-care treatment option, the present invention now offers a treatment solution wherein the radiotherapy is preferably given via an external beam at a total dose of ionizing radiation that is equal to or less than 85%, preferably, equal to or less than 80%, even more preferably, equal to or less than 75% 74%, 73%, 72%, 71% or 70%, of the total dose delivered in the standard-of-care treatment involving radiotherapy. In a particular embodiment, the external beam radiotherapy may be given as 2 Gy per fraction (five days per week) and at a total dose of ionizing radiation that is equal to or less than 56.1 Gy, preferably equal to or less than 52.8 Gy or 51 Gy, more preferably equal to or less than 50 Gy, even more preferably equal to or less than 49.1 Gy, 48 Gy or 42 Gy (compared to the total dose delivered in the standard-of-care treatment involving RT given at a total dose of 60 Gy or 66 Gy in 30 or 33 daily fractions). This reduced ionizing radiation dose can typically be delivered as a stand-alone treatment, or in combination with chemotherapy or any other relevant systemic modality as appreciated by the skilled person.

According to an embodiment of the invention, the patient has stage III or IV NSCLC and is ineligible for the standard-of-care treatment involving administration of cytotoxic agent (for example cis-platin) combined with RT (usually at a total dose of 60 or 66 Gy).

Moreover, the physical manifestations of radiation-induced lung injury can include a nonproductive cough, dyspnea with exertion, occasional low-grade fever and chest pain (which may be pleuritic or substernal and may represent pleuritis), esophageal pathology or rib fracture. Radiotherapy of the lungs may lead to a subacute inflammatory-driven reaction known as pneumonitis. Pneumonitis typically occurs between 2 and 6 months after radiotherapy to the lungs and is related to the mean lung dose, the proportion of the lung volume receiving ≥20 Gy of radiation (the so-called V20) and patient factors such as emphysema. [cf. Dirk De Ruysscher et al. Radiotherapy Toxicity. Nature Reviews, 2019, 5]. Therefore, the present invention now proposes a therapeutic solution without equivalent to those patients identified as unable/ineligible or at high risk to intolerance to the standard-of-care involving RT, due to a reduction of the total dose of RT compatible with the treatment of these patients, i.e., rendering the treatment possible in these patients due to a reduction of treatment toxicity, while maintaining therapeutic efficacy.

Also, for patients with operable locally advanced esophageal cancer (cT3-T4 or cN1-3 MO), preoperative treatment is indicated [cf. Table 1: I, A]. Patients with locally advanced squamous cell carcinoma benefit from preoperative chemotherapy or, even to a greater extent, from preoperative chemoradiotherapy (CRT), with higher rates of complete tumor resection and better local tumor control and survival [cf. Table 1: I, A]. Currently, for patients with squamous cell esophageal cancer, weekly administration of carboplatin (area under the curve of 2 mg/ml/min) and paclitaxel (50 mg/m²) for 5 weeks and concurrent RT (41.4 Gy in 23 fractions, 5 days per week), followed by surgery, is a typical therapeutic solution recommended as a contemporary standard-of-care [cf. Table 1: I, A] [cf. F. Lordick et al. Oesophageal cancer: ESMO Clinical Practice Guidelines for diagnosis, treatment and follow-up. Annals of Oncology 27 (Supplement 5): v50-v57, 2016; NCCN Guidelines, Esophageal and Esophagogastric Junction Cancer, Version 1.2020].

Typically, when considering these patients (or patients' population) diagnosed with locally advanced esophageal cancers and unable/ineligible to receive, or at high risk to intolerance to the standard-of-care combined concomitant chemoradiation, the present invention now offers a therapeutic solution without equivalent where radiotherapy is given via an external beam at a total dose of ionizing radiations that is equal to or less than 85%, preferably, equal to or less than 80%, even more preferably, equal to or less than 75%, 74%, 73%, 72; 71% or 70%, of the total dose delivered in the standard-of-care treatment involving radiotherapy. In a particular aspect, the external beam radiotherapy is given (to a patient having an operable locally advanced esophageal cancer tumor, preferably a squamous cell esophageal cancer tumor) as 1.8 or 2 Gy per fraction (five days per week) and at a total dose of ionizing radiation that is equal to or less than 35.2 Gy, preferably, equal to or less than 33.1 Gy or 31.1 Gy, more preferably, equal to or less than 30 Gy, even more preferably, equal to or less than 29 Gy (compared to the total dose delivered in the standard-of-care treatment involving RT given at a total dose of 41.4 Gy in 23 fractions 5 days per week). This reduced ionizing radiation dose can typically be delivered as a stand-alone treatment, or in combination with chemotherapy or any other relevant systemic modality as appreciated by the skilled person.

According to an embodiment of the invention, the patient has stage III or IV operable esophageal cancer and is ineligible for the standard-of-care treatment involving administration of cytotoxic agents (for example, the combination of carboplatin and paclitaxel) combined with RT (usually, at a total dose of 41.4 Gy).

Moreover, radiation-induced epithelial injury can cause damage to the skin and breakdown of mucosal membranes. In the mucosa, radiation-induced loss of stem cells from the basal layer interferes with the replacement of cells in the superficial mucosal layers when they are lost through normal physiological sloughing. The subsequent denuding of the epithelium results in mucositis, that may occur following irradiation of esophagus can be painful and interfere with oral intake and nutrition. [cf. Dirk De Ruysscher et al. Radiotherapy Toxicity. Nature Reviews, 2019, 5]. Therefore, the present invention now proposes a therapeutic solution without equivalent to those patients identified as unable/ineligible to receive or at high risk of intolerance to the standard-of-care involving RT. This therapeutic solution provides a reduction of the total dose of RT compatible with the treatment of these patients, i.e., rendering the treatment possible in these patients due to a reduction of treatment toxicity, while maintaining therapeutic efficacy.

Also, for patients diagnosed with glioblastoma multiforme (GBM), concomitant and adjuvant temozolomide (TMZ) chemotherapy in addition to radiotherapy significantly improved median, 2- and 5-year survival in a large, randomized trial, and is considered as the current standard-of-care for these patients up to age 70 years [cf. Table 1: I, A]. TMZ is administered daily (7 days a week) during radiotherapy and then for 5 days every 4 weeks for six cycles as maintenance (adjuvant) treatment after the end of irradiation. The RT recommended dose is classically 60 Gy in 2 Gy fraction or 59.4 Gy in 1.8 Gy fractions [cf. R. Stupp et al. High-grade glioma: ESMO Clinical Practice Guidelines for diagnosis, treatment and follow-up. Annals of Oncology 25 (Supplement 3): iii93-iii101, 2014; NCCN Guidelines, Central Nervous System Cancers, version 1.2020].

Typically, when considering these patients (or patients' population) diagnosed with glioblastoma and unable or at high risk to receive the combined concomitant chemoradiation given as the standard-of-care treatment option, the present invention now offers a therapeutic solution without equivalent where radiotherapy is given via an external beam at a total dose of ionizing radiations that is equal to or less than 85%, preferably equal to or less than 80%, even more preferably equal to or less than 75%, 74%, 73%, 72%, 71% or 70%, of the total dose delivered in the standard-of-care treatment involving radiotherapy. In a particular aspect, the external beam radiotherapy can now be given as 1.8 or 2 Gy per fraction (five days per week) and at a total dose of ionizing radiation that is equal to or less than 51 Gy, preferably, equal to or less than 48 Gy, more preferably, equal to or less than 42 Gy (compared to the total dose delivered in the standard-of-care treatment involving RT given at a total dose of 60 Gy, or 59.4 Gy in 30 or 33 fractions 5 days per week). This reduced ionizing radiation dose can typically be delivered as a stand-alone treatment, or in combination with chemotherapy or any other relevant systemic modality, as appreciated by the skilled person.

According to an embodiment of the invention, the patient glioblastoma is ineligible for the standard-of-care treatment involving administration of concomitant and adjuvant temozolomide (TMZ) chemotherapy combined with RT (typically at a total dose of 60 Gy given in 2 Gy fractions, or 59.4 Gy given in 1.8 Gy fractions).

Moreover, acute radiotherapy effects after brain radiotherapy include the development of oedema, whereas later adverse effects include radionecrosis (tissue death associated with irradiation). Other adverse outcomes after brain radiotherapy include pseudo-progression of the tumor (i.e., an increase of lesion size related to treatment, following by tumor response), brain inflammation and tumor progression. Neurocognitive impairment is observed in 25-65% of patients who have received radiotherapy to the brain [cf. Dirk De Ruysscher et al. Radiotherapy Toxicity. Nature Review, 2019, 5]. Therefore, the present invention now offers a therapeutic solution without equivalent to those patients unable/ineligible, or at high risk of intolerance to undergo the standard-of-care involving RT. This is due to a reduction of the total dose of RT compatible with the treatment of these patients, i.e., rendering the treatment possible in these patients thanks to a reduction of treatment toxicity, while maintaining therapeutic efficacy.

The herein described invention can also be applied to other cancer indications and clinical stages, typically in a human patient suffering from a cancer and unable to receive a standard-of-care treatment involving RT, wherein the cancer is typically a skin cancer, including a malignant neoplasm associated to AIDS, a melanoma or a squamous cancer; a central nervous system cancer including for example a brain, cerebellum, pituitary, spinal cord, brainstem, eye or orbit cancer; a head and neck cancer; a lung cancer; a breast cancer; a gastrointestinal cancer such as a liver and a hepatobiliary tract cancer, a colon, a rectum and/or an anal cancer, a stomach cancer, a pancreas cancer, an esophagus cancer; a male genitourinary cancer such as for example a prostate, testis, penis and/or urethra cancer; a gynecologic cancer such as for example a uterine cervix, endometrium, ovary, fallopian tube, vagina and/or vulvar cancer; an adrenal and/or retroperitoneal cancer; a sarcoma of bone and soft tissue regardless its localization; and a pediatric cancer such as for example a Wilm's cancer, a neuroblastoma, a central nervous system cancer, a Ewing's sarcoma, etc.

In an embodiment of the invention, the patient receives a total dose between 45 and 60 Gy when the total RT dose in the standard-of-care treatment is 70 Gy. For example, the patient may receive 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57 or 57.5 Gy, when the total RT dose in the stand of care treatment is 70 Gy. In a preferred embodiment of the invention, the patient may receive a total dose of between 45 and 55 Gy, for example 45, 45.5, 46, 46.5, 47, 47.5, 48, 48.5, 49, 49.5, 50, 50.5, 51, 51.5, 52, 52.5, 53, 53.5, 54, 54.5 or 55 Gy (the total

RT dose in the standard-of-care treatment being 70 Gy). In a more preferred embodiment of the invention, the patient may receive a total dose of between 47 and 52 Gy (the total dose in the stand of care treatment being 70 Gy).

In an embodiment of the invention, the patient receives a total dose between 40 and 55 Gy (the total RT dose in the standard-of-care treatment being 60 Gy). For example, the patient may receive less than 54 Gy, preferably a dose equal to or less than 53 GY, 52.8 Gy or 51 Gy, more preferably a dose equal to or less than 50 Gy, even more preferably a dose equal to or less than 49.1 Gy, 48 Gy or 42 Gy.

In an embodiment of the invention, the patient receives a total radiation dose of between 29 and 37 Gy (the total RT dose in the standard-of-care treatment being 41 Gy). For example, the patient may receive 37, 35, 33, 31 or 29 Gy.

Nanoparticles and/or Aggregates of Nanoparticles

Size

In the spirit of the invention, the term “nanoparticle” refers to a product, in particular a synthetic product, with a size in the nanometer range, typically between about 1 nm and about 1000 nm, preferably between about 1 nm and about 500 nm, even more preferably between about 1 and about 100 nm.

The term “aggregate of nanoparticles” refers to an assemblage of nanoparticles.

The size of the nanoparticle and/or aggregates of nanoparticle can typically be measured by Electron Microscopy (EM) technics, such as transmission electron microscopy (TEM) or cryo-TEM, as well known by the skilled person. The size of at least 100 nanoparticles and/or aggregates of nanoparticles is typically measured and the median size of the population of nanoparticles and/or aggregates of nanoparticles is reported as the size of the nanoparticle and/or aggregate of nanoparticles.

Shape

As the shape of the nanoparticles and/or aggregates of nanoparticles can influence its “biocompatibility”, nanoparticles and/or aggregates of nanoparticles having a quite homogeneous shape are preferred. For pharmacokinetic reasons, nanoparticles and/or aggregates of nanoparticles being essentially spherical, round or ovoid in shape are thus preferred. Such a shape also favors the nanoparticle and/or aggregates of nanoparticles interaction with, or uptake by, cells.

Composition/Structure

In a preferred aspect herein described the nanoparticles and/or aggregates of nanoparticles of the present invention comprise more than 30%, preferably more than 40%, 50%, 60%, 70% or 80% by weight of at least one chemical element having an atomic number (Z) between 20 and 83, preferably between 40 and 83, even more preferably between 57 and 83.

The determination of the percentage of chemical element having an atomic number (Z) between 20 and 83 is performed on the nanoparticles and/or aggregates nanoparticles having no biocompatible surface coating as herein below described (i.e., prior any biocompatible surface coating of the nanoparticle and/or aggregate of nanoparticles). The determination of the percentage of chemical element having an atomic number (Z) between 20 and 83 is typically performed using an Inductively Coupled Plasma (ICP) source, such as an ICP-MS (Mass Spectroscopy) tool which is a type of mass spectrometry that uses the Inductively Coupled Plasma to ionize the sample or an ICP-OES (Optical Emission Spectroscopy) tool which is a type of emission spectroscopy that uses the Inductively Coupled Plasma to produce excited atoms and ions that emit electromagnetic radiation at wavelengths characteristic of a particular element. The results of the quantification are typically expressed as a percentage (%) by weight of the chemical element per weight of the nanoparticle and/or aggregate of nanoparticles (i.e. % w/w).

As a theoretical example, if the nanoparticle and/or aggregate of nanoparticles is made of hafnium oxide (HfO₂), the theoretical percentage (%) by weight of the chemical element hafnium (Hf) (Z_{Hf}=72), per weight of the nanoparticle and/or aggregates of nanoparticles (hafnium oxide (HfO₂)) is equal to 85% (% w/w):
178.49/210.49×100=85% (% w/w), where 178.49 is the molecular weight of Hf element and 210.49 is the molecular weight of HfO₂ material.

Any experimental quantification of a chemical element constituting the nanoparticle and/or aggregate of nanoparticles can be expressed as a percentage by weight of this chemical element per weight of nanoparticle and/or aggregate of nanoparticle as herein above presented in the context of a theoretical calculation.

In a preferred aspect, the nanoparticle and/or aggregate of nanoparticles comprises at least one, for example two or three distinct, chemical element(s) selected from La, Ce, Pr, Nd, Pm, Sm, Eu, Gd, Tb, Dy, Ho, Er, Tm, Yb, Lu, Hf, Ta, W, Re, Os, Ir, Pt, Au and Bi.

In a preferred aspect, the nanoparticle and/or aggregate of nanoparticles is an inorganic nanoparticle and/or aggregate of nanoparticles, i.e., the material constituting the nanoparticle and/or the aggregate of nanoparticles is an inorganic material typically selected from an hydroxide, an oxohydroxide, an oxide, a metal, a tungstate, a sulfide and any mixture thereof.

The inorganic material of the nanoparticle and/or aggregate of nanoparticles preferably has a theoretical (bulk) density of at least 7 g/cm³ and may be selected from any material exhibiting this property and identified in the table from Physical Constants of Inorganic Compounds appearing on page 4-43 in Handbook of Chemistry and Physics (David R. Lide Editor-in-Chief, 88th Edition 2007-2008).

In a preferred aspect, the inorganic nanoparticle and/or aggregate of nanoparticles comprises at least one, for example two or three distinct, chemical element(s) selected from La, Ce, Pr, Nd, Pm, Sm, Eu, Gd, Tb, Dy, Ho, Er, Tm, Yb, Lu, Hf, Ta, W, Re, Os, Ir, Pt, Au and Bi. When the inorganic nanoparticle and/or aggregate of nanoparticles comprises at least one, for example two or three distinct, chemical element(s) with Z between 20 and 83, at least one chemical element is present within the nanoparticle and/or aggregate of nanoparticles at a level corresponding to more than 30%, preferably more than 40%, 50%, 60%, 70% or 80% by weight per weight of nanoparticle and/or aggregate of nanoparticles (% w/w).

When the inorganic material constituting the nanoparticle and/or aggregate of nanoparticles is a metal oxide, this metal oxide is advantageously selected from Titanium oxide (TiO₂), Cerium (IV) oxide (CeO₂), Neodymium (III) oxide (Nd₂O₃), Samarium (III) oxide (Sm₂O₃), Europium (III) oxide (Eu₂O₃), Gadolinium (III) oxide (Gd₂O₃), Terbium (III) oxide (Tb₂O₃), Dysprosium (III) oxide (Dy₂O₃), Holmium oxide (Ho₂O₃), Erbium oxide (Er₂O₃), Thulium (III) oxide (Tm₂O₃), Ytterbium oxide (Yb₂O₃), Lutetium oxide (lu₂O₃), Hafnium (IV) oxide (HfO₂), Tantalum (V) oxide (Ta₂O₅), Rhenium (IV) oxide (ReO₂) and Bismuth (III) (Bi₂O₃). In the context of the present invention, a mixture of inorganic metal oxides can also be used to prepare the nanoparticles and/or aggregates of nanoparticles of the invention. The inorganic metal oxide(s) may be doped with La, Ce, Pr, Nd, Pm, Sm, Eu, Gd, Tb, Dy, Ho, Er, Tm, Yb and/or Lu.

When the inorganic material constituting the nanoparticle and/or aggregate of nanoparticles is a metal, this metal is advantageously selected from gold (Au), platinum (Pt), palladium (Pd), tin (Sn), tantalum (Ta), ytterbium (Yb), zirconium (Zr), hafnium (Hf), terbium (Tb), thulium (Tm), cerium (Ce), dysprosium (Dy), erbium (Er), europium (Eu), holmium (Ho), lanthanum (La), neodymium (Nd), praseodymium (Pr), lutetium (Lu) and mixtures thereof. In the context of the present invention, a mixture of an inorganic metal oxide and of a metal can also be used to prepare the nanoparticles and/or aggregates of nanoparticles of the invention.

When the inorganic material constituting the nanoparticle is a sulfide, this sulfide is preferably silver sulfide (Ag₂S).

When the inorganic material constituting the nanoparticle is a tungstate, this tungstate is preferably a calcium tungstate (CaWO₄).

In a preferred aspect, the nanoparticles are selected from a HfO₂ (hafnium oxide) nanoparticle, a Au (gold) nanoparticle, a ReO₂ (rhenium oxide) nanoparticle and a mixture thereof.

Biocompatible Coating

In a particular aspect of the description, each of the nanoparticles and/or aggregates of nanoparticles of the present invention further comprises a biocompatible surface coating.

In a preferred aspect, each of the nanoparticle and/or aggregate of nanoparticles used in the context of the present invention can be coated with a biocompatible material, preferably with an agent exhibiting stealth property. Indeed, when the nanoparticles and/or aggregates of nanoparticles of the present invention are administered to a subject via the intravenous (IV) route, a biocompatible coating with an agent exhibiting stealth property is particularly advantageous to optimize the biodistribution of the nanoparticles and/or aggregates of nanoparticles. Such coating is responsible for the so called “stealth property” of the nanoparticle or of the aggregate of nanoparticles. The agent exhibiting stealth properties may be an agent displaying a steric group. Such a group may be selected for example from polyethylene glycol (PEG); polyethylenoxide; polyvinylalcohol; polyacrylate; polyacrylamide (poly(N-isopropylacrylamide)); polycarbamide; a biopolymer; a polysaccharide such as for example dextran, xylan and cellulose; collagen; and a zwitterionic compound such as for example polysulfobetain; etc.

In another preferred aspect, each of the nanoparticle and/or aggregate of nanoparticles can be coated with an agent allowing interaction with a biological target. Such an agent can typically bring a positive or a negative charge on the nanoparticle ‘s or aggregate of nanoparticles’ surface. This charge can be easily determined by zeta potential measurements, typically performed on nanoparticles and/or aggregates of nanoparticles suspensions the concentration of which vary between 0.2 and 10 g/L, the nanoparticles being suspended in an aqueous medium with a pH comprised between 6 and 8.

An agent forming a positive charge on the surface of the nanoparticles or the aggregate of nanoparticles can be for example aminopropyltriethoxisilane or polylysine. An agent forming a negative charge on the surface of the nanoparticles or the aggregate of nanoparticles can be for example a phosphate (for example a polyphosphate, a metaphosphate, a pyrophosphate, etc.), a carboxylate (for example citrate or dicarboxylic acid, in particular succinic acid) or a sulphate.

A full biocompatible coating of the nanoparticle or the aggregate of nanoparticles may be advantageous, in particular for an intravenous (IV) administration in the human patient, in order to avoid interaction of the surface of the nanoparticles or aggregate of nanoparticles with any recognition element (macrophage, opsonins, etc.).

The biocompatible coating allows in particular, the nanoparticle's and/or aggregate of nanoparticles' stability in a fluid, typically in a physiological fluid (such as blood, plasma, serum, etc.), any isotonic media or physiologic media, for example media comprising glucose (5%) and/or NaCl (0.9), which is required for a pharmaceutical administration.

Stability may be confirmed by dry extract quantification using a drying oven and measured in a suspension of nanoparticles and/or aggregates of nanoparticles prior and after filtration, typically on a 0.22 μm or 0.45 μm filter. Advantageously, the coating preserves the integrity of the nanoparticle and/or aggregate of nanoparticles in vivo, ensures or improves the biocompatibility thereof, and facilitates an optional functionalization thereof (for example with spacer molecules, biocompatible polymers, targeting agents, proteins, etc.).

Targeting

A particular nanoparticle and/or aggregate of nanoparticles as herein described can further comprise a targeting agent allowing its interaction with a recognition element present on a target cell, typically on a cancer cell. Such a targeting agent typically acts once the nanoparticles and/or aggregates of nanoparticles are accumulated on the target site, typically on the tumor site. The targeting agent can be any biological or chemical structure displaying affinity for molecules present in the human or animal body. For instance, it can be a peptide, oligopeptide or polypeptide, a protein, a nucleic acid (DNA, RNA, SiRNA, tRNA, miRNA, etc.), a hormone, a vitamin, an enzyme, the ligand of a molecule expressed by a pathological cell, in particular the ligand of a tumor antigen, hormone receptor, cytokine receptor or growth factor receptor. Said targeting agent can be for example in the group consisting in LHRH, EGF, a folate, anti-B-FN antibody, E-selectin/P-selectin, anti-IL-2Ra antibody, GHRH, etc.

Composition

Also herein described is a pharmaceutical composition comprising nanoparticles and/or aggregates of nanoparticles such as herein above described and a pharmaceutically acceptable carrier, vehicle or support.

The pharmaceutical composition herein described is, in a preferred aspect herein described, for use in the treatment of cancer in a human patient, typically in a human patient unable/ineligible, or at high risk, to undergo/of intolerance to a standard-of-care treatment involving radiotherapy (RT), wherein the nanoparticles and/or aggregates of nanoparticles preferably advantageously comprise more than 30% by weight of at least one, for example two or three distinct, chemical element(s) having an atomic number (Z) between 20 and 83, preferably between 40 and 83, wherein the treatment of cancer involves typically a step of administering the nanoparticles and/or aggregates of nanoparticles to said patient, and a step of exposing the patient who has been administered with the nanoparticles and/or aggregates of nanoparticles to a total dose of ionizing radiations that is typically equal to or less than 85%, preferably equal to or less than 80%, more preferably equal to or less than 79%, 78%, 77%, 76% or 75%, even more preferably equal to or less than 74%, 73%, 72%, 71% or 70%, of the total dose delivered in the standard-of-care treatment involving radiotherapy.

The composition can be in the form of a solid, liquid (typically nanoparticles and/or aggregates of nanoparticles in suspension), aerosol, gel, paste, and the like. Preferred compositions are in a liquid or a gel form. Particularly preferred compositions are in liquid form.

The carrier which is employed can be any classical support for the skilled person, such as for example a saline, isotonic, sterile, buffered solution, or a non-aqueous vehicle solution and the like.

The composition can also comprise stabilizers, sweeteners, surfactants, polymers and the like. It can be formulated for example as ampoule, aerosol, bottle, tablet, capsule, by using techniques of pharmaceutical formulation known by the skilled person.

Generally, the composition, in liquid or gel form, comprises between about 0.05 g/L and about 450 g/L of nanoparticles and/or aggregates of nanoparticles, for example between about 0.05 g/L and about 250 g/L of nanoparticles and/or aggregates of nanoparticles, preferably at least about 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 85 g/L, 90 g/L, 95 g/L, 100 g/L, 150 g/L, 200 g/L, 250 g/L, 300 g/L, 350 g/L, or 400 g/L of nanoparticles and/or aggregates of nanoparticles.

The concentration of nanoparticles and/or aggregates of nanoparticles in the composition can be measured by dry extract. A dry extract is ideally measured following a drying step of the suspension comprising the nanoparticles in a drying oven.

Administration Route

The nanoparticles and/or aggregates of nanoparticles of the invention can be administered to the subject using different possible routes such as local (intra-tumoral (IT), intra-arterial (IA)), subcutaneous, intravenous (IV), intra-dermic, airways (inhalation), intraperitoneal, intramuscular, intra-articular, intra-thecal, intra-ocular or oral route (per os), preferably using IT, IV or IA.

Repeated injections or administrations of nanoparticles and/or aggregates of nanoparticles can be performed, when appropriate.

Radiotherapy Sources

In a typical aspect herein described, nanoparticles and/or aggregates of nanoparticles are to be administered to the subject to be treated and said subject is then to be exposed to ionizing radiations. These ionizing radiations are typically selected from X-rays, gamma-rays, electrons and protons. Preferred ionizing radiations are X-rays.

In a preferred aspect, the nanoparticle and/or aggregate of nanoparticles or subject who has been administrated with the nanoparticle and/or aggregate of nanoparticles, is to be exposed to ionizing radiations.

RT comprises multiple different treatment modalities, including external beam therapy (encompassing photons, electrons, protons and other particles) and internal/surface treatment (brachytherapy and radiopharmaceuticals). In a preferred aspect herein described, ionizing radiation is selected from X-rays, gamma-rays, electrons and protons.

As indicated herein above, appropriate radiations are preferably ionizing radiations and can advantageously be selected from the group consisting of X-Rays, gamma-Rays, electron beams (electrons), ion beams (such as protons) and radioactive isotopes or radioisotopes emissions. X-Rays are particularly preferred ionizing radiations.

Ionizing radiations are typically of about 2 KeV to about 25 000 KeV, in particular of about 2 KeV to about 6000 KeV (i.e. 6 MeV) (LINAC source).

In general, and in a non-restrictive manner, the following X-Rays can be applied in different circumstances to excite the herein described particles:
Superficial X-Rays of 2 to 50 keV: to excite nanoparticles near the skin surface (penetration of a few millimeters);
X-Rays of 50 to 150 keV: in diagnostic but also in therapy;
X-Rays (ortho voltage) of 200 to 500 keV which can penetrate a biological tissue thickness of 6 cm;
X-Rays (mega voltage) of 1000 keV to 25 000 keV.

Radioactive isotopes can alternatively be used as ionizing radiation (typically in the context of curie therapy or brachytherapy). In particular, Iodine 1-125 (t 1/2=60.1 days), Palladium Pd-103 (t 1/2=17 days), Cesium Cs-137, Strontium Sr-89 (t 1/2=50.5 days), Samarium Sm-153 (t 1/2=46.3 hours), and Iridium Ir-192, can advantageously be used.

Electron beams may also be used as ionizing radiation and have an energy typically comprised between 4 MeV and 25 MeV.

In a particular aspect, a specific monochromatic irradiation source can be used for selectively generating

X-rays radiation at energy close to, or corresponding to, the desired X-ray absorption edge of the atoms constituting the metallic material of the nanoparticles and/or aggregates of nanoparticles selected for use in the context of the invention.

Preferentially, ionizing radiations are X-rays obtained from Linear Accelerator (LINAC) or are protons.

Combination

In a particular aspect of the invention, the herein described use and/or therapeutic treatment further comprises a step of administering at least one distinct therapeutic agent or drug, in particular, an immunotherapeutic agent, to the subject, preferably, to the human patient, either simultaneously or separately from the herein described nanoparticles or aggregates of nanoparticles.

The terms “immunotherapeutic agent” herein designates typically any molecule, drug, cell or cell-based vaccine, oncolytic virus, DNA-based vaccine, peptide-based vaccine, toll-like receptor agonist, vesicle derived from a cell as well as any combination thereof capable of boosting the immune system of a subject and recognized as such by the skilled person. The molecule or drug, in particular the immunotherapeutic agent, can for example be selected from a monoclonal antibody, a cytokine, and a combination thereof.

The agent or drug can be for example an indoleamine 2,3-dioxygenase (IDO) inhibitor such as the 1-methyl-D-tryptophan.

In a preferred aspect herein described, the monoclonal antibody inhibits the CTLA-4 molecule or the interaction between PD-1 and its ligands. The monoclonal antibody is advantageously selected from anti-CTLA-4, anti-PD-1, anti-PD-L1, and anti-PD-L2. The monoclonal antibody can for example be selected from ipilimumab, tremelimumab, nivolumab, prembolizumab, pidilizumab and lambrolizumab.

In another preferred aspect herein described, the monoclonal antibody enhances CD27 signaling, CD 137 signaling, OX-40 signaling, GITR signaling and/or MHCII signaling, and/or activate CD40. The monoclonal antibody can for example be selected from dacetuzumab, lucatumumab and urelumab. In a further aspect herein described, the monoclonal antibody inhibits TGF-β signaling or KIR signaling. The monoclonal antibody can for example be selected from fresolimumab and lirilumab.

The cytokine can be advantageously selected from the granulocyte-macrophage colony stimulating factor (GM-CSF), a FMS-related tyrosine kinase 3 ligand (FLT3L), IFN-α, IFN-α2b, IFNγ, IL2, IL-7, IL-10 and IL-15.

In another preferred aspect herein described, the immunotherapeutic agent is an immunocytokine, for example the immunocytokine L19-IL2 [cf. Nicolle H. Rekers Radiotherapy and Oncology 2015].

The kind of cell which can be used in the context of the present invention as an immunotherapeutic agent is typically an immune cell presenting or sensitized to a tumor antigen, preferably a tumor antigen specific to the cancer to be treated, such as a dendritic cell or a T-cell; a cell secreting an immunogenic molecule; a dead tumor cell or a dying tumor cell undergoing an immunogenic cell death, i.e. a cell expressing CRT and/or producing HMGB1 and/or producing ATP in a ICD typical amount, for example a dying or dead-tumor cell which has been exposed to radiotherapy. The cell can be an autologous cell or an allogeneic cell. The cell is preferably an autologous cell isolated from the subject to be treated. The dead- or dying-tumor cell can be a tumor mature cell or a tumor stem cell.

A toll-like receptor agonist which can be used in the context of the present invention can be advantageously selected from a TLR 2/4 agonist, a TRL 7 agonist, a TRL 7/8 agonist and a TRL 9 agonist. The toll-like receptor agonist can be selected for example from imiquimod, bacillus Calmette-Guerin and monophosphoryl lipid A.

A preferred combination of immunotherapeutic agents usable in the context of the present invention can combine for example at least two members of the following list: a cytokine, a monoclonal antibody, a Toll-like receptor agonist and a peptide-based vaccine.

The terms “therapeutic agent” herein typically designates an agent used in a treatment of cancer such as a biological compound, a small molecule targeted therapeutic, or a cytotoxic compound.

A biological compound is for instance an antibody, preferably a monoclonal antibody (“mAb”) such as Alemtuzumab, Brentuximab Vedotin, Catumaxoma, Denosumab, Gemtuzumab ozogamicin, Ibritumomab tiuxetan, Pertuzumab, Ofatumumab, bevacizumab, rituximab, trastuzumab, cetuximab, panatimumab or tositumomab.

A small molecule targeted therapeutic/agent generally inhibits an enzymatic domain on a mutated, overexpressed, or otherwise critical protein (potential target in the context of cancer treatment) within the malignant cells. Some therapeutic agents include those that target cell division (for example a aurora-kinase inhibitor or a cyclin-dependent-kinase inhibitor), or any other biological mechanism such as protein turnover and chromatin modification (for example a histone-deacetylase inhibitor). The small molecule targeted therapeutic/agent can for example be selected from imatinib, rapamycin, gefitinib, erlotinib, sorafenib, sunitinib, nilotinib, dasatinib, lapatinib, bortezomib and atorvastatin.

A cytotoxic compound is for example a DNA-modifying agent such as an anthracyclin (for example dexamethasone, daunorubicin, idarubicin or methotrexate) or an antimitotic agent (spindle poison such as vincristine or vinblastine); a taxane such as docetaxel, larotaxel, cabazitaxel, paclitaxel (PG-paclitaxel and DHA-paclitaxel), ortataxel, tesetaxel or taxoprexin; gemcitabine; etoposide; mitomycine C; an alkylating agent (for example melphalan or temozolomide); a platin based component such as oxaliplatin or carboplatin; and a TLR (Toll-like receptor)-3 ligand. The compound may be a prodrug. The prodrug (for instance capecitabine or irinotecan) is metabolized in its active form in vivo to produce its expected therapeutic effect.

A typical cytotoxic compound may be any chemotherapeutic agent known by the multidisciplinary (oncology) team and considered by the team as relevant for the particular cancer and/or subject.

The effect of the herein described invention has been demonstrated in the clinic and is illustrated in Example 3, below, taken from interim results of ongoing phase I clinical trial NCT01946867, which are disclosed herein for the first time. The administration of a composition comprising nanoparticle aggregates according to the invention followed by irradiation with X-rays, leads to complete response (CR) according to the Guidelines RESCIST v1.1 (Eisenhauer, E. A., (2009) New response evaluation criteria in solid tumours: Revised RECIST guideline (version 1.1) Eur. J. Cancer, 45, pp 228-247) in a specific group of patients (with locally advanced SCC of the oral Cavity or oropharynx) at a significantly lower total dose of radiation than that used in the standard-of-care treatment. It should be noted that these patients are RT responders. Surprisingly, a CR could be achieved in a significant number of patients at a radiation total dose approximately 71% of standard-of-care radiation dose. Thus, the data indicates that, the nanoparticles and nanoparticles aggregates of the invention allow one to successfully treat a specific group of cancer patients using a total dose of radiation that is significantly less than that of the standard-of-care total dose. In certain embodiment of the invention, one may successfully treat some cancer patients with a total dose of radiation that is less than 85%, preferably, less than 80%, more preferably, less than 75% and most preferably less than 72% of the standard-of-care radiation total dose.

The examples, which follow, illustrate the invention without limiting the scope thereof.

### EXAMPLES

### Example 1. Synthesis of HfO₂ Nanoparticles

A Tetramethylammonium hydroxide (TMAOH) solution is added to 40 g of HfCl₄ solution. Addition of TMAOH solution is performed until the pH of the final suspension reaches a pH comprised between 7 and 13. A white precipitate is obtained.

The precipitate is further transferred in an autoclave and heated at a temperature comprised between 120° C. and 300° C. to perform crystallization. After cooling, the suspension is washed with de-ionized water.

A peptization step, is performed to obtain a stable suspension of nanoparticles or aggregates of nanoparticles.

Suspension of Sodium hexametaphosphate is then added to the peptized solution and the pH of the suspension is adjusted to a pH comprised between 6 and 8.

The concentration of HfO₂ nanoparticles and/or aggregates of nanoparticles in the composition is typically measured by dry extract following a drying step of the above suspension in a drying oven.

For in vivo experiments, a formulation step using glucose 5% is typically performed.

### Example 2. Synthesis of Au Nanoparticles

Gold nanoparticles are obtained by reduction of gold chloride with sodium citrate in aqueous solution. Protocol was adapted from G. Frens Nature Physical Science 241 (1973) 21.

In a typical experiment, HAuCl₄ solution is heated to boiling. Subsequently, sodium citrate solution is added. The resulting solution is maintained under boiling for an additional period of 5 minutes.

The nanoparticle size is typically adjusted from 15 up to 105 nm by carefully modifying the citrate versus gold precursor ratio.

The as prepared gold nanoparticles suspensions are then concentrated using an ultrafiltration device (Amicon stirred cell model 8400 from Millipore) with a 30 kDa cellulose membrane.

The resulting suspensions are ultimately filtered through a 0.22 μm cutoff membrane filter (PES membrane from Millipore) under laminar hood and stored at 4° C.

Gold content is determined by ICP-MS and expressed as [Au] in g/L.

Particle size is determined using Transmission Electronic Microscopy (TEM) by counting more than 100 particles, taking the longest nanoparticle dimension for size measurement.

### Example 3. Treatment of Locally Advanced Stage III or IVAHNSCC in Patients Who are Ineligible for Standard-of-Care Treatment in a Phase I Clinical Trial NCT01946867

Standard-of-care for stage III or IVA HNSCC of the oral cavity or oropharynx is cis-platin and irradiation with a total dose of 70 Gy. In this ongoing phase I trial, patients with stage III or IVA (American Joint committee on cancer (AJCC Guidelines, 7^{th} edition, 2010) HNSCC of the oral cavity or oropharynx, aged more than or equal to 70 years old, or more than or equal to 65 years old and ineligible to receive cisplatin, amenable to RT with curative intent, are treated with a composition according to an embodiment of the invention. Interim data is available for 35 patients.

Patients received a composition comprising 5% glucose, 53.3 g/L crystalline hafnium oxide nanoparticles bearing a negatively charged compatible coating, as a single intra tumoral (IT) injection, followed by activation by intensity modulated radiation therapy (IMRT) delivered as 2 Gy fractions (5 fractions per week) over 7 weeks. Patients were injected with a volume of composition corresponding to 22% of the tumor volume. The response rate was measured after 50 Gy and, again, after 70 Gy total radiation dose.

Interim results indicate that, surprisingly, about one quarter of patients have experienced complete response (CR) at a total dose of 50 Gy (corresponding to 71.4% of the standard-of-care treatment), after only five weeks of treatment, rather than seven weeks, which is the time to administer the full 70 Gy standard-of-care dose. These patients reached their best overall response (BOR), which was CR, already at 50 Gy.

At the second measurement time point—11 weeks into the study and—4 weeks after RT ended, a further 34% of patients reached a BOR of either CR, or partial response (PR). These patients may indeed have reached their BOR before the full standard-of care radiation dose of 70 Gy was given. The remaining patients (approximately 30%) either experienced stable disease (SD) or progressive disease (PD) at both 50 Gy and 70 Gy. Thus, the patients with PD did not respond fully to RT.

These unexpected results demonstrate the efficacy of the innovative treatment in this fragile and difficult-to-treat patient population.

It is now possible to successfully treat this patient subpopulation with radiation doses that are considerably lower than those of standard-of-care treatments, following administration of the nanoparticles of the invention.

1-18. (canceled)

19. A method for treating cancer in a human patient ineligible to undergo a standard-of-care treatment involving radiotherapy (RT) or in a patient at high risk of intolerance to a standard-of-care treatment involving RT, wherein the method comprises a step of administering nanoparticles and/or aggregates of nanoparticles to said patient, the nanoparticles and/or aggregates of nanoparticles comprising more than 30% by weight of at least one chemical element having an atomic number (Z) between 20 and 83, and a step of exposing the patient who has been administered with the nanoparticles and/or aggregates of nanoparticles to a total dose of ionizing radiation that is equal to or less than 85% of the total dose delivered in the standard-of-care treatment involving RT for said cancer.

20. The method according to claim 19, wherein the patient is ineligible to undergo a standard of care treatment involving RT because said treatment is contraindicated for said patient.

21. The method according to claim 19, wherein the patient fulfills at least one of the following criteria:
a) is vulnerable or frail according to the comprehensive geriatric assessment (CGA),
b) has a high comorbidity index score as evaluated using the Adult Comorbidity Evaluation 27 (ACE-27) and the Charlson Comorbidity Index (CCI),
c) has grade 2 or more organ dysfunction based on the National Cancer Institute Common Toxicity Criteria (NCI CTC),
d) has poor functional status as measured as an ECOG performance status of 2 or more, or as an equivalent Karnofski performance status,
e) is exposed to more than 4 distinct prescribed drugs,
f) is elderly and evaluated as “fit”, according to part or full components evaluation of CGA, but presents cumulative exposure to risk factors for being intolerant to treatment,
g) has a locally advanced tumor observed at clinical staging,
h) has an inoperable stage IIIA or TIM Non-Small Cell Lung Carcinoma (NSCLC) tumor at clinical staging.

22. The method according to claim 19, wherein the human patient has a locally advanced tumor at clinical staging.

23. The method according to claim 19, wherein the cancer is selected from a skin cancer, a central nervous system cancer, a head and neck cancer, a lung cancer, a breast cancer, a gastrointestinal cancer, a male genitourinary cancer, a gynecologic cancer, an adrenal and/or retroperitoneal cancer, a sarcoma of bone and soft tissue, and a pediatric cancer.

24. The method according to claim 23, wherein the patient has a locally advanced stage III or IV squamous cell carcinoma of the head and neck (SCCHN) at clinical staging, and receives a total dose of radiation equal to or less than 59.5 Gy delivered as a stand-alone treatment or in combination with chemotherapy or any other relevant systemic modality.

25. The method according to claim 24, wherein the patient receives a total dose of radiation equal to or less than: a) 56 Gy, b) 52.5 Gy, or c) 50 Gy, delivered as a stand-alone treatment or in combination with chemotherapy or any other relevant systemic modality.

26. The method according to claim 23, wherein the patient has a stage IIIA or stage IIIB non-small cell Lung Carcinoma (NSCLC) tumor evaluated as inoperable at clinical staging, and receives a total dose of radiation equal to or less than 56.1 Gy delivered as a stand-alone treatment or in combination with chemotherapy or any other relevant systemic modality.

27. The method according to claim 26, wherein the patient receives a total dose of radiation equal to or less than: a) 52.8 Gy, b) 51 Gy, c) 50 Gy, d) 49.1 Gy, e) 48 Gy, or f) 42 Gy, delivered as a stand-alone treatment or in combination with chemotherapy or any other relevant systemic modality.

28. The method according to claim 23, wherein the patient has an operable locally advanced esophageal cancer tumor and receives a preoperative total dose of radiation equal to or less than 35.2 Gy delivered as a stand-alone treatment or in combination with chemotherapy or any other relevant systemic modality.

29. The method according to claim 28, wherein the patient has a squamous cell esophageal cancer tumor.

30. The method according to claim 28, wherein the patient receives a preoperative total dose of radiation equal to or less than: a) 33.1 Gy, b) 31.1 Gy, c) 30 Gy, or d) 29 Gy delivered as a stand-alone treatment or in combination with chemotherapy or any other relevant systemic modality.

31. The method according to claim 23, wherein the patient has glioblastoma and receives a total dose of radiation equal to or less than 51 Gy delivered as a stand-alone treatment or in combination with chemotherapy or any other relevant systemic modality.

32. The method according to claim 31, wherein the patient receives a total dose of radiation equal to or less than: a) 48 Gy, or b) 42 Gy delivered as a stand-alone treatment or in combination with chemotherapy or any other relevant systemic modality.

33. The method according to claim 19, wherein each nanoparticle comprises at least one chemical element selected from La, Ce, Pr, Nd, Pm, Sm, Eu, Gd, Tb, Dy, Ho, Er, Tm, Yb, Lu, Hf, Ta, W, Re, Os, Ir, Pt, Au and Bi.

34. The method according to claim 19, wherein the nanoparticles are inorganic nanoparticles.

35. The method according to claim 19, wherein each nanoparticle and/or aggregate of nanoparticles further comprises a biocompatible surface coating.

36. The method according to claim 19, wherein the nanoparticles are selected from a HfO₂ nanoparticle, a Au nanoparticle, a ReO₂ nanoparticle and any mixture thereof.

37. The method according to claim 19, wherein the ionizing radiation is selected from X-rays, gamma-rays, electrons and protons.

38. The method according to claim 19, wherein the treatment further comprises administering at least one distinct therapeutic agent to the human patient, either simultaneously or separately.

39. The method according to claim 38, wherein the at least one distinct therapeutic agent is an immunotherapeutic agent selected from a monoclonal antibody, a cytokine and a combination thereof.

40. The method according to claim 38, wherein the at least one distinct therapeutic agent is selected from a biological compound, a small molecule targeted therapeutic, a cytotoxic compound and a combination thereof.

41. A method for treating cancer in a human patient ineligible to undergo a standard-of-care treatment involving RT or in a patient at high risk of intolerance to a standard-of-care treatment involving RT, wherein the nanoparticles and/or aggregates of nanoparticles comprise more than 30% by weight of at least one chemical element having an atomic number (Z) between 20 and 83, wherein the method comprises a step of administering to said patient a pharmaceutical composition comprising nanoparticles and/or aggregates of nanoparticles and a pharmaceutically acceptable carrier or support, the nanoparticles and/or aggregates of nanoparticles comprising more than 30% by weight of at least one chemical element having an atomic number (Z) between 20 and 83, and a step of exposing the patient who has been administered with the nanoparticles and/or aggregates of nanoparticles to a total dose of ionizing radiation that is equal to or less than 85% of the total dose delivered in the standard-of-care treatment involving RT for said cancer.
